(19) United States
(12) Patent Application Publication
(10) Pub. No.: US 2026/0066038 A1
(43) Pub. Date: Mar. 5, 2026

(54) TECHNIQUES FOR COMPOSITIONAL PROTEIN GENERATION

(71) Applicant: NVIDIA CORPORATION, Santa Clara, CA (US)

(72) Inventors: Karsten KREIS, Vancouver (CA); Tomas GEFFNER, San Jose, CA (US); Bowen JING, Cambridge, MA (US); Hannes Axel STAERK, Cambridge, MA (US); Arash VAHDAT, San Mateo, CA (US)

(21) Appl. No.: 19/092,860

(22) Filed: Mar. 27, 2025

Related U.S. Application Data
(60) Provisional application No. 63/688,650, filed on Aug. 29, 2024.

(51) Int. Cl. G16B 15/20; G16B 40/20; G16B 45/00

(52) U.S. Cl. CPC G16B 15/20; G16B 40/20; G16B 45/00

(57) ABSTRACT

The disclosed method for generating proteins includes generating, using a trained machine learning model, a first protein based on a three-dimensional (3D) representation of a spatial layout for the first protein, where generating the first protein comprises applying cross-attention between one or more first tokens associated with the 3D representation and one or more second tokens associated with a second protein.

### CROSS-REFERENCE TO RELATED APPLICATIONS

This application claims priority benefit of the United States Provisional patent application titled, “TECHNIQUES FOR GENERATING COMPOSITIONAL PROTEIN STRUCTURES,” filed on Aug. 29, 2024 and having Ser. No. 63/688,650. The subject matter of this related application is hereby incorporated herein by reference.

### BACKGROUND

### Technical Field

Embodiments of the present disclosure relate generally to computer science, artificial intelligence, and machine learning, and more specifically, to techniques for compositional protein generation.

### Description of the Related Art

Advances in machine learning have enabled the development of machine learning models capable of generating novel protein designs. Proteins are sequences of amino acids, also referred to as “residues,” that fold into complex structures depending on the particular amino acids in those sequences. Proteins serve essential functions in biological systems, including catalyzing chemical reactions, providing structural support, and facilitating cellular communication. Designing new proteins with specific properties can lead to breakthroughs in medicine, biotechnology, and materials science, among other things.

Conventional machine learning models, particularly deep learning architectures, have been trained to learn patterns from large datasets of known protein structures and sequences. Once trained, the machine learning models can be used to generate new proteins, including proteins that optimize desired properties such as stability, binding affinity, or catalytic activity that are specified as objectives that guide the generation process. For example, transformer-based models that are trained on protein sequences and structures have been used to generate functional proteins by capturing complex dependencies between amino acids.

One drawback of the above approach is conventional machine learning models for generating proteins do not permit user control over the three-dimensional (3D) spatial layouts of those proteins, such as the locations of alpha helices and/or beta sheets. Oftentimes, users having domain expertise will understand that certain spatial layouts are likely to impart desired properties on a protein. However, conventional machine learning models generate proteins according to automatically learned patterns, without allowing users to control the spatial layouts of those proteins. Accordingly, the proteins that are generated by conventional machine learning models can lack desired properties or otherwise be suboptimal for desired purposes.

As the foregoing illustrates, what is needed in the art are more effective techniques for generating proteins.

### SUMMARY

One embodiment of the present disclosure sets forth a computer-implemented method for generating proteins. The method includes generating, using a trained machine learning model, a first protein based on a three-dimensional (3D) representation of a spatial layout for the first protein. Generating the first protein comprises applying cross-attention between one or more first tokens associated with the 3D representation and one or more second tokens associated with a second protein.

Other embodiments of the present disclosure include, without limitation, one or more computer-readable media including instructions for performing one or more aspects of the disclosed techniques as well as one or more computing systems for performing one or more aspects of the disclosed techniques.

At least one technical advantage of the disclosed techniques relative to the prior art is the disclosed techniques permit users to control the 3D spatial layouts of proteins that are generated by a trained machine learning model. In particular, the 3D spatial layouts can be controlled using 3D ellipsoid representations that are informative enough to control the generation of diverse proteins, while being human-interpretable and easy to construct, such as through sketches of ellipsoids in the ellipsoid representations. As the function of a protein depends on the structure of the protein, being able to explicitly control the 3D spatial layouts of generated proteins according to techniques disclosed herein permits the generated proteins to exhibit desired properties to a higher degree than proteins that are generated according to prior art approaches. These technical advantages represent one or more technological improvements over prior art approaches.

### BRIEF DESCRIPTION OF THE DRAWINGS

So that the manner in which the above recited features of the various embodiments can be understood in detail, a more particular description of the inventive concepts, briefly summarized above, may be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1 illustrates a block diagram of a computer-based system configured to implement one or more aspects of the various embodiments;

FIG. 2 is a more detailed illustration of the machine learning server of FIG. 1, according to various embodiments;

FIG. 3 is a more detailed illustration of the computing device of FIG. 1, according to various embodiments;

FIG. 4 is a more detailed illustration of the protein generating application of FIG. 1, according to various embodiments;

FIG. 5 is a more detailed illustration of the iterative integration module of FIG. 4, according to various embodiments;

FIG. 6 is a more detailed illustration of the model trainer of FIG. 1, according to various embodiments;

FIG. 7 illustrates how an exemplar ellipsoid representation can be generated from a known protein, according to various embodiments;

FIG. 8A illustrates exemplar proteins generated based on ellipsoid representations generated by a statistical model, according to various embodiments;

FIG. 8B illustrates exemplar proteins generated based on user-specified ellipsoid representations, according to various embodiments;

FIG. 9 is a flow diagram of method steps for training a protein generative model, according to various embodiments;

FIG. 10 is a flow diagram of method steps for generating proteins using a trained protein generative model, according to various embodiments; and

FIG. 11 is a flow diagram of method steps for performing an update block within a trained protein generative model, according to various embodiments.

### DETAILED DESCRIPTION

In the following description, numerous specific details are set forth to provide a more thorough understanding of the various embodiments. However, it will be apparent to one skilled in the art that the inventive concepts may be practiced without one or more of these specific details.

### General Overview

Embodiments of the present disclosure provide techniques for generating proteins conditioned on three-dimensional (3D) representations. In some embodiments, a 3D representation includes one or more shapes, such as one or more ellipsoids, specifying the locations of one or more annotated portions of a protein, such as the locations of secondary structures (e.g., alpha helices and/or beta sheets) within the protein or the locations of portions of the protein having certain functionalities or other properties. A user can specify a 3D representation to use for generating a protein. Alternatively, a protein generating application can automatically generate a 3D representation using a statistical model. In the case of a 3D representation that includes ellipsoids, the statistical model can randomly sample means and covariance matrices for a number of ellipsoids, while penalizing configurations in which ellipsoids overlap. The protein generating application generates a protein conditioned on a user-specified or automatically generated 3D representation by iteratively integrating a vector field defined by a neural network that is learned via a flow matching technique. The flow matching technique learns a flow that can be described through a differential equation or continuous time Markov chain, which the protein generating application can then numerically solve in a step-wise manner by sampling the neural network. The generated protein includes a sequence of residues and a structure conforming to the 3D representation. The iterative integration of the vector field includes, for multiple time steps, processing a current protein and the 3D representation using a trained protein generative model to generate an updated protein. By performing multiple such iterative steps, a protein that begins as random noise can be transformed into a protein that conforms to the 3D representation. The protein generative model is the neural network that includes, among other things, an invariant cross attention that allows tokens corresponding to the protein to attend to tokens corresponding to the 3D representation. In some embodiments, generating a protein can include interpolating a conditional vector field that is conditioned on a 3D representation and an unconditional vector field based on a guidance parameter.

To train the protein generative model, a model trainer performs 3D segmentation on a number of proteins, such as proteins from a library of proteins, and the model trainer generates 3D representations based on the 3D segmentations. For example, in some embodiments, the model trainer can segment the proteins into secondary structures or based on the functionality or other properties of portions of the proteins, fit Gaussians to the segmented portions of the proteins, and convert the Gaussians to ellipsoids using, for example, a predefined Mahalanobis distance to define boundaries of the ellipsoids. Then, the model trainer can train the protein generative model using a flow matching technique and the 3D representations and corresponding proteins as training data.

The techniques for generating molecules have many real-world applications. For example, those techniques could be applied to generate proteins that are useful in medicine, biotechnology, and materials science, among other things.

The above examples are not in any way intended to be limiting. As persons skilled in the art will appreciate, as a general matter, the techniques for generating proteins can be implemented in any suitable application.

### System Overview

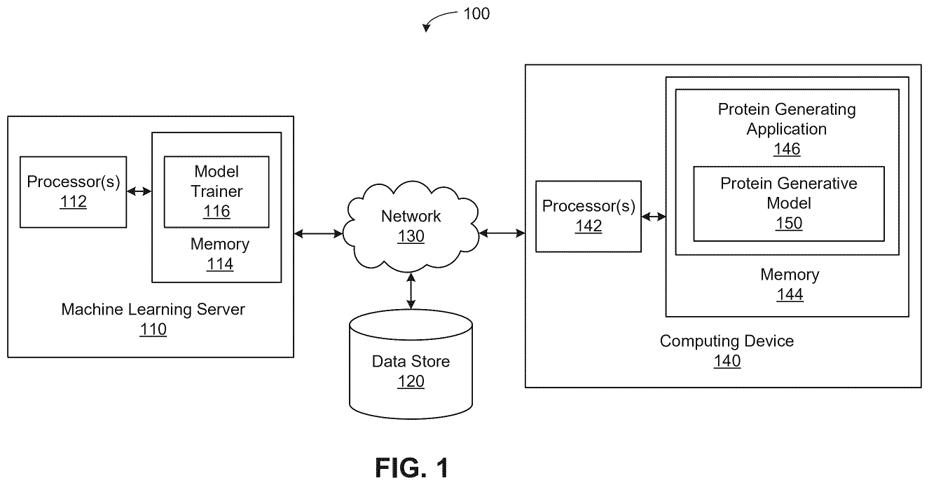

FIG. 1 illustrates a block diagram of a computer-based system 100 configured to implement one or more aspects of at least one embodiment. As shown, the system 100 includes a machine learning server 110, a data store 120, and a computing device 140 in communication over a network 130, which can be a wide area network (WAN) such as the Internet, a local area network (LAN), a cellular network, and/or any other suitable network.

As shown, a model trainer 116 executes on one or more processors 112 of the machine learning server 110 and is stored in a system memory 114 of the machine learning server 110. The processor 112 receives user input from input devices, such as a keyboard or a mouse. In operation, the one or more processors 112 may include one or more primary processors of the machine learning server 110, controlling and coordinating operations of other system components. In particular, the processor(s) 112 can issue commands that control the operation of one or more graphics processing units (GPUs) (not shown) and/or other parallel processing circuitry (e.g., parallel processing units, deep learning accelerators, etc.) that incorporates circuitry optimized for graphics and video processing, including, for example, video output circuitry. The GPU(s) can deliver pixels to a display device that can be any conventional cathode ray tube, liquid crystal display, light-emitting diode display, and/or the like.

The system memory 114 of the machine learning server 110 stores content, such as software applications and data, for use by the processor(s) 112 and the GPU(s) and/or other processing units. The system memory 114 can be any type of memory capable of storing data and software applications, such as a random-access memory (RAM), a read-only memory (ROM), an erasable programmable read-only memory (EPROM or Flash ROM), or any suitable combination of the foregoing. In some embodiments, a storage (not shown) can supplement or replace the system memory 114. The storage can include any number and type of external memories that are accessible to the processor 112 and/or the GPU. For example, and without limitation, the storage can include a Secure Digital Card, an external Flash memory, a portable compact disc read-only memory (CD-ROM), an optical storage device, a magnetic storage device, and/or any suitable combination of the foregoing.

The machine learning server 110 shown herein is for illustrative purposes only, and variations and modifications are possible without departing from the scope of the present disclosure. For example, the number of processors 112, the number of GPUs and/or other processing unit types, the number of system memories 114, and/or the number of applications included in the system memory 114 can be modified as desired. Further, the connection topology between the various units in FIG. 1 can be modified as desired. In some embodiments, any combination of the processor(s) 112, the system memory 114, and/or GPU(s) can be included in and/or replaced with any type of virtual computing system, distributed computing system, and/or cloud computing environment, such as a public, private, or a hybrid cloud system.

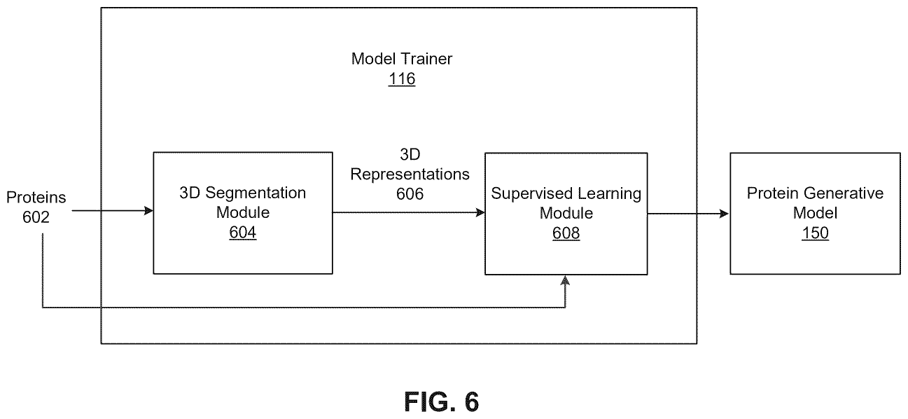

In some embodiments, the model trainer 116 is configured to train one or more machine learning models, including a protein generative model 150 that is trained to generate proteins conditioned on user-specified 3D representations of layouts of the proteins. Techniques for training the protein generative model 150 are discussed in greater detail below in conjunction with FIGS. 6-7 and 9. Training data and/or trained machine learning models, including the protein generative model 150, can be stored in the data store 120, or elsewhere. In some embodiments, the data store 120 can include any storage device or devices, such as fixed disc drive(s), flash drive(s), optical storage, network attached storage (NAS), and/or a storage area-network (SAN). Although shown as accessible over the network 130, in at least one embodiment the machine learning server 110 can include the data store 120.

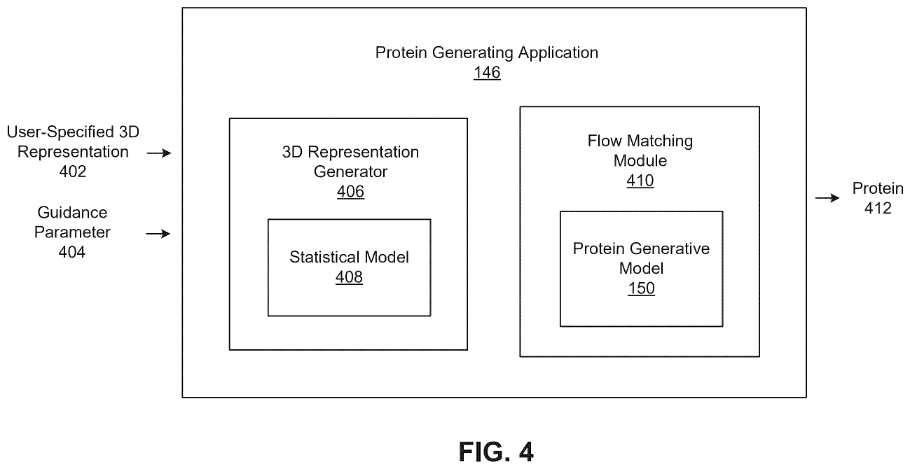

As shown, a protein generating application 146 that uses the trained protein generative model 150 is stored in a memory 144, and executes on processor(s) 142, of the computing device 140. The memory 144 and the processor(s) 142 may be similar to the memory 114 and the processors 112, respectively, of the machine learning server, described above. The protein generating application 146 can use the trained protein generative model 150 to generate proteins that conform to user-specified 3D representations of layouts of the proteins, as discussed in greater detail below in conjunction with FIGS. 4-5, 8, and 10-11.

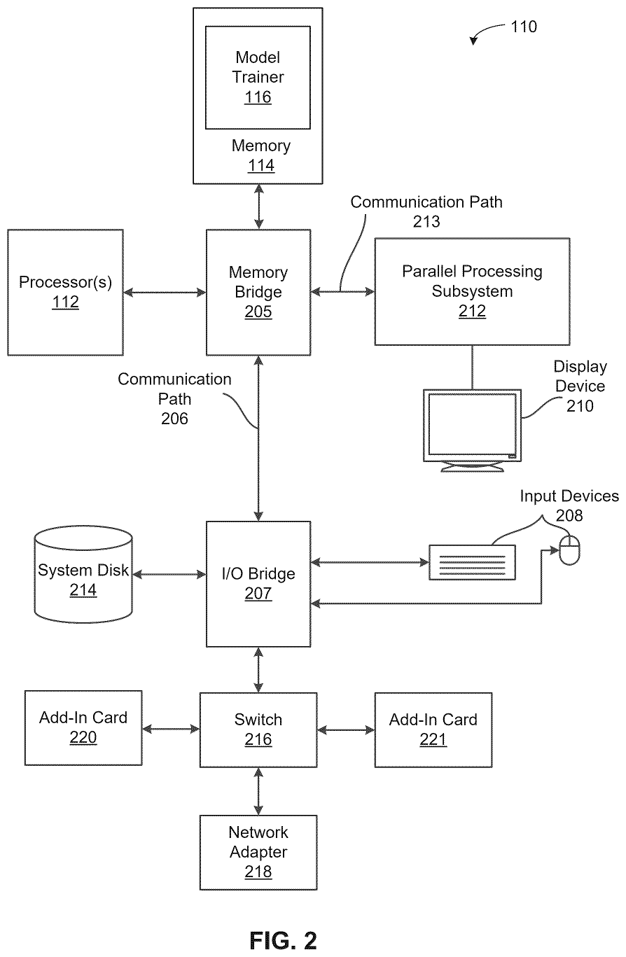

FIG. 2 is a block diagram illustrating the machine learning server 110 of FIG. 1 in greater detail, according to various embodiments. The machine learning server 110 may include any type of computing system, including, without limitation, a server machine, a server platform, a desktop machine, a laptop machine, a hand-held/mobile device, a digital kiosk, an in-vehicle infotainment system, and/or a wearable device. In some embodiments, the machine learning server 110 is a server machine operating in a data center or a cloud computing environment that provides scalable computing resources as a service over a network. In some embodiments, the machine learning server 110 can include one or more similar components as the machine learning server 110.

In various embodiments, the machine learning server 110 includes, without limitation, the processor(s) 112 and the memory(ies) 114 coupled to a parallel processing subsystem 212 via a memory bridge 205 and a communication path 213. Memory bridge 205 is further coupled to an I/O (input/output) bridge 207 via a communication path 206, and I/O bridge 207 is, in turn, coupled to a switch 216.

In one embodiment, I/O bridge 207 is configured to receive user input information from optional input devices 208, such as a keyboard, mouse, touch screen, sensor data analysis (e.g., evaluating gestures, speech, or other information about one or more uses in a field of view or sensory field of one or more sensors), and/or the like, and forward the input information to the processor(s) 112 for processing. In some embodiments, the machine learning server 110 may be a server machine in a cloud computing environment. In such embodiments, machine learning server 110 may not include input devices 208, but may receive equivalent input information by receiving commands (e.g., responsive to one or more inputs from a remote computing device) in the form of messages transmitted over a network and received via the network adapter 218. In some embodiments, switch 216 is configured to provide connections between I/O bridge 207 and other components of the machine learning server 110, such as a network adapter 218 and various add-in cards 220 and 221.

In some embodiments, I/O bridge 207 is coupled to a system disk 214 that may be configured to store content and applications and data for use by processor(s) 112 and parallel processing subsystem 212. In one embodiment, system disk 214 provides non-volatile storage for applications and data and may include fixed or removable hard disk drives, flash memory devices, and CD-ROM (compact disc read-only-memory), DVD-ROM (digital versatile disc-ROM), Blu-ray, HD-DVD (high-definition DVD), or other magnetic, optical, or solid state storage devices. In various embodiments, other components, such as universal serial bus or other port connections, compact disc drives, digital versatile disc drives, film recording devices, and the like, may be connected to I/O bridge 207 as well.

In various embodiments, memory bridge 205 may be a Northbridge chip, and I/O bridge 207 may be a Southbridge chip. In addition, communication paths 206 and 213, as well as other communication paths within machine learning server 110, may be implemented using any technically suitable protocols, including, without limitation, AGP (Accelerated Graphics Port), HyperTransport, or any other bus or point-to-point communication protocol known in the art.

In some embodiments, parallel processing subsystem 212 comprises a graphics subsystem that delivers pixels to an optional display device 210 that may be any conventional cathode ray tube, liquid crystal display, light-emitting diode display, and/or the like. In such embodiments, the parallel processing subsystem 212 may incorporate circuitry optimized for graphics and video processing, including, for example, video output circuitry. Such circuitry may be incorporated across one or more parallel processing units (PPUs), also referred to herein as parallel processors, included within the parallel processing subsystem 212.

In some embodiments, the parallel processing subsystem 212 incorporates circuitry optimized (e.g., that undergoes optimization) for general purpose and/or compute processing. Again, such circuitry may be incorporated across one or more PPUs included within parallel processing subsystem 212 that are configured to perform such general purpose and/or compute operations. In yet other embodiments, the one or more PPUs included within parallel processing subsystem 212 may be configured to perform graphics processing, general purpose processing, and/or compute processing operations. System memory 114 includes at least one device driver configured to manage the processing operations of the one or more PPUs within parallel processing subsystem 212. In addition, the system memory 114 includes the model trainer 116. Although described herein primarily with respect to the model trainer 116, techniques disclosed herein can also be implemented, either entirely or in part, in other software and/or hardware, such as in the parallel processing subsystem 212.

In various embodiments, parallel processing subsystem 212 may be integrated with one or more of the other elements of FIG. 2 to form a single system. For example, parallel processing subsystem 212 may be integrated with processor(s) 112 and other connection circuitry on a single chip to form a system on a chip (SoC).

In some embodiments, processor(s) 112 includes the primary processor of machine learning server 110, controlling and coordinating operations of other system components. In some embodiments, the processor(s) 112 issues commands that control the operation of PPUs. In some embodiments, communication path 213 is a PCI Express link, in which dedicated lanes are allocated to each PPU. Other communication paths may also be used. The PPU advantageously implements a highly parallel processing architecture, and the PPU may be provided with any amount of local parallel processing memory (PP memory).

It will be appreciated that the system shown herein is illustrative and that variations and modifications are possible. The connection topology, including the number and arrangement of bridges, the number of processor(s) 202, and the number of parallel processing subsystems 212, may be modified as desired. For example, in some embodiments, system memory 114 could be connected to the processor(s) 112 directly rather than through memory bridge 205, and other devices may communicate with system memory 114 via memory bridge 205 and processor(s) 112. In other embodiments, parallel processing subsystem 212 may be connected to I/O bridge 207 or directly to processor(s) 112, rather than to memory bridge 205. In still other embodiments, I/O bridge 207 and memory bridge 205 may be integrated into a single chip instead of existing as one or more discrete devices. In certain embodiments, one or more components shown in FIG. 2 may not be present. For example, switch 216 could be eliminated, and network adapter 218 and add-in cards 220, 221 would connect directly to I/O bridge 207. Lastly, in certain embodiments, one or more components shown in FIG. 2 may be implemented as virtualized resources in a virtual computing environment, such as a cloud computing environment. In particular, the parallel processing subsystem 212 may be implemented as a virtualized parallel processing subsystem in at least one embodiment. For example, the parallel processing subsystem 212 may be implemented as a virtual graphics processing unit(s) (vGPU(s)) that renders graphics on a virtual machine(s) (VM(s)) executing on a server machine(s) whose GPU(s) and other physical resources are shared across one or more VMs.

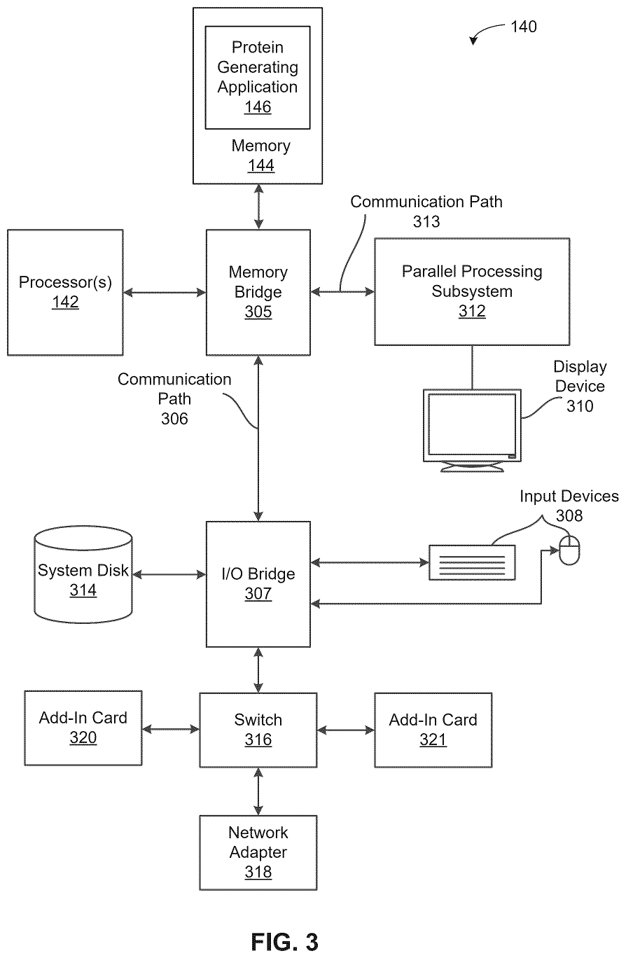

FIG. 3 is a block diagram illustrating the computing device 140 of FIG. 1 in greater detail, according to various embodiments. The computing device 140 may include any type of computing system, including, without limitation, a server machine, a server platform, a desktop machine, a laptop machine, a hand-held/mobile device, a digital kiosk, an in-vehicle infotainment system, and/or a wearable device. In some embodiments, the computing device 140 is a server machine operating in a data center or a cloud computing environment that provides scalable computing resources as a service over a network. In some embodiments, the machine learning server 110 can include one or more similar components as the computing device 140.

In various embodiments, the computing device 140 includes, without limitation, the processor(s) 142 and the memory(ies) 144 coupled to a parallel processing subsystem 312 via a memory bridge 305 and a communication path 313. Memory bridge 305 is further coupled to an I/O (input/output) bridge 307 via a communication path 306, and I/O bridge 307 is, in turn, coupled to a switch 316.

In one embodiment, I/O bridge 307 is configured to receive user input information from optional input devices 308, such as a keyboard, mouse, touch screen, sensor data analysis (e.g., evaluating gestures, speech, or other information about one or more uses in a field of view or sensory field of one or more sensors), and/or the like, and forward the input information to the processor(s) 142 for processing. In some embodiments, the computing device 140 may be a server machine in a cloud computing environment. In such embodiments, computing device 140 may not include input devices 308, but may receive equivalent input information by receiving commands (e.g., responsive to one or more inputs from a remote computing device) in the form of messages transmitted over a network and received via the network adapter 318. In some embodiments, switch 316 is configured to provide connections between I/O bridge 307 and other components of the computing device 140, such as a network adapter 318 and various add-in cards 320 and 321.

In some embodiments, I/O bridge 307 is coupled to a system disk 314 that may be configured to store content and applications and data for use by processor(s) 142 and parallel processing subsystem 312. In one embodiment, system disk 314 provides non-volatile storage for applications and data and may include fixed or removable hard disk drives, flash memory devices, and CD-ROM (compact disc read-only-memory), DVD-ROM (digital versatile disc-ROM), Blu-ray, HD-DVD (high-definition DVD), or other magnetic, optical, or solid state storage devices. In various embodiments, other components, such as universal serial bus or other port connections, compact disc drives, digital versatile disc drives, film recording devices, and the like, may be connected to I/O bridge 307 as well.

In various embodiments, memory bridge 305 may be a Northbridge chip, and I/O bridge 307 may be a Southbridge chip. In addition, communication paths 306 and 313, as well as other communication paths within computing device 140, may be implemented using any technically suitable protocols, including, without limitation, AGP (Accelerated Graphics Port), HyperTransport, or any other bus or point-to-point communication protocol known in the art.

In some embodiments, parallel processing subsystem 312 comprises a graphics subsystem that delivers pixels to an optional display device 310 that may be any conventional cathode ray tube, liquid crystal display, light-emitting diode display, and/or the like. In such embodiments, the parallel processing subsystem 312 may incorporate circuitry optimized for graphics and video processing, including, for example, video output circuitry. Such circuitry may be incorporated across one or more parallel processing units (PPUs), also referred to herein as parallel processors, included within the parallel processing subsystem 312.

In some embodiments, the parallel processing subsystem 312 incorporates circuitry optimized (e.g., that undergoes optimization) for general purpose and/or compute processing. Again, such circuitry may be incorporated across one or more PPUs included within parallel processing subsystem 312 that are configured to perform such general purpose and/or compute operations. In yet other embodiments, the one or more PPUs included within parallel processing subsystem 312 may be configured to perform graphics processing, general purpose processing, and/or compute processing operations. System memory 144 includes at least one device driver configured to manage the processing operations of the one or more PPUs within parallel processing subsystem 312. In addition, the system memory 144 includes the protein generating application 146. Although described herein primarily with respect to the protein generating application 146, techniques disclosed herein can also be implemented, either entirely or in part, in other software and/or hardware, such as in the parallel processing subsystem 312.

In various embodiments, parallel processing subsystem 312 may be integrated with one or more of the other elements of FIG. 3 to form a single system. For example, parallel processing subsystem 312 may be integrated with processor 142 and other connection circuitry on a single chip to form a system on a chip (SoC).

In some embodiments, processor(s) 142 includes the primary processor of computing device 140, controlling and coordinating operations of other system components. In some embodiments, the processor(s) 142 issues commands that control the operation of PPUs. In some embodiments, communication path 313 is a PCI Express link, in which dedicated lanes are allocated to each PPU. Other communication paths may also be used. The PPU advantageously implements a highly parallel processing architecture, and the PPU may be provided with any amount of local parallel processing memory (PP memory).

It will be appreciated that the system shown herein is illustrative and that variations and modifications are possible. The connection topology, including the number and arrangement of bridges, the number of processor(s) 302, and the number of parallel processing subsystems 312, may be modified as desired. For example, in some embodiments, system memory 144 could be connected to the processor(s) 142 directly rather than through memory bridge 305, and other devices may communicate with system memory 144 via memory bridge 305 and processor 142. In other embodiments, parallel processing subsystem 312 may be connected to I/O bridge 307 or directly to processor 142, rather than to memory bridge 305. In still other embodiments, I/O bridge 307 and memory bridge 305 may be integrated into a single chip instead of existing as one or more discrete devices. In certain embodiments, one or more components shown in FIG. 3 may not be present. For example, switch 316 could be eliminated, and network adapter 318 and add-in cards 320, 321 would connect directly to I/O bridge 307. Lastly, in certain embodiments, one or more components shown in FIG. 3 may be implemented as virtualized resources in a virtual computing environment, such as a cloud computing environment. In particular, the parallel processing subsystem 312 may be implemented as a virtualized parallel processing subsystem in at least one embodiment. For example, the parallel processing subsystem 312 may be implemented as a virtual graphics processing unit(s) (vGPU(s)) that renders graphics on a virtual machine(s) (VM(s)) executing on a server machine(s) whose GPU(s) and other physical resources are shared across one or more VMs.

### Compositional Protein Generation

FIG. 4 is a more detailed illustration of the protein generating application 146 of FIG. 1, according to various embodiments. As shown, the protein generating application 146 includes, without limitation, a three-dimensional (3D) representation generator 406 and a iterative integration module 410. The 3D representation generator 406 includes, without limitation, a statistical model 408. The iterative integration module 410 includes, without limitation, the protein generative model 150.

In operation, the protein generating application 146 can receive (e.g., via a user interface) as input a user-specified 3D representation 402 and a guidance parameter 404. The user-specified 3D representation 402 indicates the 3D spatial layout of a protein. For example, in some embodiments, the user-specified 3D representation 402 can be a 3D ellipsoid representation that includes (1) one or more ellipsoids specifying the locations of one or more portions of a protein, such as the locations of secondary structures (e.g., alpha helices and/or beta sheets) within the protein or the locations of portions of the protein that have certain functionalities or other properties (e.g., functionally relevant sites such as binding sites for a ligand of interest, electron density, etc.), and (2) annotations of the secondary structures, functionalities, and/or other properties associated with different ellipsoids. Experience has shown that 3D ellipsoid representations are informative enough to control the generation of diverse proteins, while being human-interpretable and easy to construct, such as through sketches of ellipsoids in the 3D ellipsoid representations. Further, with 3D ellipsoid representations, users are not required to specify finer grain details, such as each residue in a protein, which may be difficult and cumbersome to specify manually. Accordingly, 3D ellipsoid representations provide an intermediate level of guidance for the generation of proteins. Alternatively, in some embodiments, the 3D representation generator 406 can automatically generate a 3D representation using the statistical model 408 if no user-specified 3D representation is received as input. In some embodiments, the 3D representation generator 406 can generate 3D ellipsoid representations by, for a set of ellipsoids associated with randomly assigned annotations, randomly sampling means for the ellipsoids from a distribution (e.g., a Gaussian distribution) and randomly sampling covariance matrices for the set of ellipsoids from another distribution (e.g., a Wishart distribution that is a distribution over plausible covariance matrices), while penalizing configurations in which ellipsoids overlap significantly.

Mathematically, a protein spatial layout that includes K ellipsoids can be defined as an unordered set E={Eₖ=(μₖ, Σₖ, fₖ, nₖ)}_{k∈{1 . . . K}}, where each ellipsoid is represented as a Gaussian with mean μₖ ∈ [symbol] ³, covariance Σᵢ ∈ [symbol] ^{3×3}, count nₖ ∈ [symbol] ⁺ and feature annotation fₖ ∈ [symbol] , where [symbol] is the application-dependent feature space. Viewed as Gaussian probability distributions, the ellipsoids do not have well-defined boundaries. However, for visualization and evaluation purposes, the ellipsoid boundary could be a surface at a certain Mahalanobis distance, such as a Mahalanobis distance of \sqrt{5} that causes 83% of the density to fall inside the surface:

$$\partial {E}_{k} = \{ x ∈ {ℝ}^{3} : \sqrt{{( x - {μ}_{k} )}^{T} {\sum }_{k}^{- 1} ( x - {μ}_{k} )} = \sqrt{5} \}$$

The spatial layout of a protein can be defined using a set of K ellipsoids, each corresponding to a semantically coherent region of the protein. Each ellipsoid can record the number of residues in the associated region, a categorical semantic feature, a position, and/or a shape in terms of the covariance matrix of the Cα coordinates in the region. 3D ellipsoid representations of protein spatial layouts offer a favorable tradeoff between a single global annotation, such as a text prompt or protein family, and more complex shape descriptors, such as meshes or voxel grids. Although described herein primarily with respect to 3D ellipsoid representations, any technically feasible representations of 3D protein spatial layouts can be used in some embodiments, such as cylinder representations, bounding boxes, voxel grids, representative points or lines, meshes, and/or the like.

In some embodiments, the 3D representation generator 406 can generate 3D ellipsoid representations by sampling synthetic ellipsoids from an additional generative model p_{θ}(E) to sample an unconditional distribution of protein structures factorized as p_{θ}(t, R, a|E)p_{θ}(E). Instead of a deep learned p_{θ}(E) that may produce layouts that are similar to the training data, a statistical model for p_{θ}(E) guarantees sampling diverse and novel layouts, which lead to more diverse and novel protein structures from p_{θ}(t, R, a|E)p_{θ}(E), properties that are crucial for protein design where the aim is commonly to produce novel designs. To generate novel ellipsoid layouts, the 3D representation generator 406 can first sample means and covariances for K ellipsoids and then assign secondary structure and residue count annotations. The model over means and covariances can be:

$$p ( {\{ ( {μ}_{k} , {\sum }_{k} ) \}}_{k = 1}^{K} ) ∝ [ {\prod }_{k = 1}^{K} 𝒩 ( {μ}_{k} , 0 , {σ}^{2} {I}_{3} ) {𝒲}_{3} ( {\sum }_{k} ; {ψ}^{2} {I}_{3} , v ) ] exp ( - U ( {\{ ( {μ}_{k} , {\sum }_{k} ) \}}_{k = 1}^{K} ) ) , U ( {\{ ( {μ}_{k} , {\sum }_{k} ) \}}_{k = 1}^{K} ) = {\sum }_{k \neq j} \frac{1}{{[ {( {μ}_{k} - {μ}_{j} )}^{T} {\sum }_{k}^{- 1} ( {μ}_{k} - {μ}_{j} ) ]}^{2}} .$$

That is, the ellipsoid means and covariances are drawn independently and identically distributed from isotropic Gaussian and Wishart distributions, respectively, and multiplied with the Boltzmann factor of an energy function that penalizes ellipsoid overlaps. Intuitively, a controls the ellipsoid's spread, 7 controls their volume, v controls their anisotropy or “roundness”, and U prevents overlaps. The energy U is a simple inverse square repulsion based on pairwise Mahalanobis distances. The 3D representation generator 406 can perform such sampling via rejection sampling, i.e., by sampling μᵢ, Σᵢ, evaluating their energy U, and rejecting with probability e^{-U}. To choose the ellipsoid annotations, each ellipsoid can first be independently annotated as α with probability γ and β with probability 1−γ. For a given choice of {α, β}, the ellipsoid volume \sqrt{det Σᵢ} strongly determines the residue count by a simple linear fit. Hence, in some embodiments, the 3D representation generator 406 can use a linear fit to assign the number of residues instead of modeling the number of residues independently.

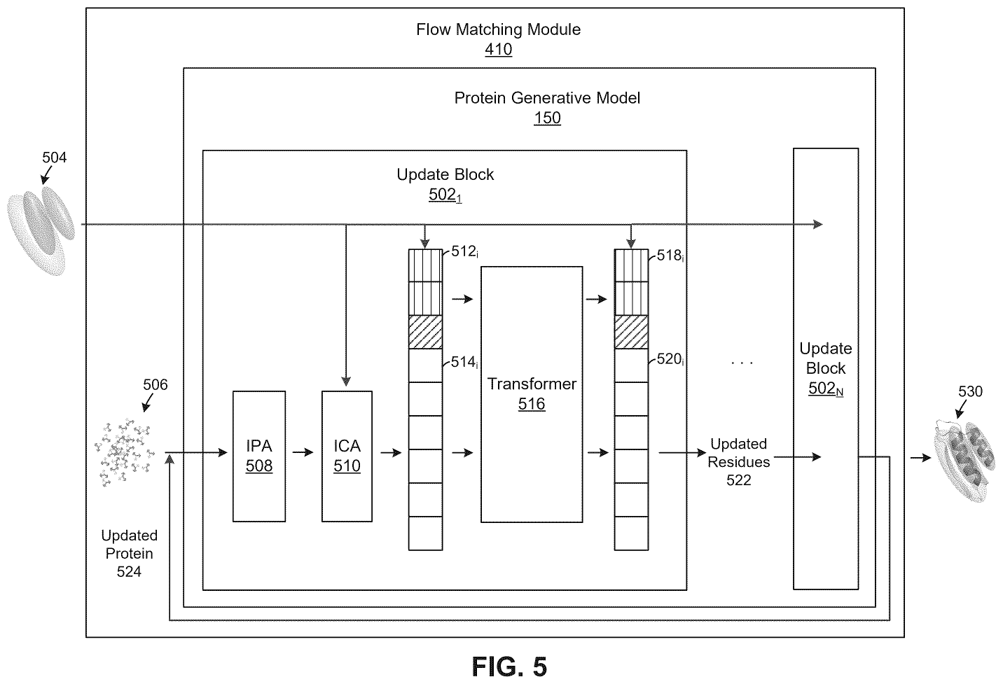

The iterative integration module 410 performs an iterative integration of a neural network-defined vector field, which is learned via a flow-matching technique, conditioned on the 3D representation (that is either the user-specified 3D representation 402 or an automatically-generated 3D representation) in order to generate a protein 412. The trained protein generative model 150 is the neural network in some embodiments. In some embodiments, the iterative integration module 410 can interpolate a conditional vector field that is conditioned on the 3D representation and an unconditional vector field that is not conditioned on the 3D representation based on a guidance parameter that indicates how to interpolate the two vector fields. In some embodiments, the iterative integration module 410 includes an invariant cross attention that allows tokens corresponding to a protein structure to attend to tokens corresponding to the 3D representation that is used as conditioning information, as discussed in greater detail below in conjunction with FIG. 5. In some embodiments, the generated protein 412 is a design of a physical protein that includes a sequence of residues and a structure that are jointly generated.

Flow matching is a generative modeling technique that allows for training continuous normalizing flows without the need for simulation, essentially learning how to transform a simple distribution into a complex data distribution by matching the flow between the two distributions through a vector field that guides the transformation process. Flow matching aims to learn a time-dependent vector field v_{θ,t} that, when integrated from a start time t=0 to t=1, transports samples from a noise distribution x₀˜p₀ to a data distribution x₁˜p₁. Although described herein primarily with respect to integrating a vector field learned via a flow matching technique as a reference example, in some embodiments, proteins can be generated using the trained protein generative model 150 in any technically feasible manner, such as using a diffusion technique.

FIG. 5 is a more detailed illustration of the iterative integration module 410 of FIG. 4, according to various embodiments. As shown, the iterative integration module 410 includes, without limitation, the protein generative model 150. The protein generative model 150 includes, without limitation, a number of update blocks 502_{1-N} (referred to herein collectively as update blocks 502 and individually as an update block 502). Update block 502₁ of update blocks 502 includes, without limitation, an invariant point attention (IPA) layer 508, an invariant cross attention (ICA) layer 510, and a transformer 516.

In operation, when generating a denoising prediction conditioned on a 3D representation, the iterative integration module 410 receives, as input, the 3D representation 504. The 3D representation 504 can be user-specified or automatically generated using the statistical model 408. The iterative integration module 410 also initializes the iterative integration of the neural network-defined vector field using a noisy protein 506. In some embodiments, the iterative integration module 410 can generate the noisy protein 506 to include residues having random positions and orientations. Each residue is an amino acid that can be included in a protein. The iterative integration module 410 performs an update step to integrate the neural network-defined vector field using the protein generative model 150, which is the neural network, and conditioned on the input 3D representation 504, to generate an updated protein 524 from a current protein, which begins from the noisy protein 506. The updated protein 524 can then be used in additional update steps to generate more updated proteins, until a stopping condition is met, such as a predefined number of update steps have been performed (e.g., 100 steps).

As described, each update step includes processing a current protein (e.g., the noisy protein 506 in the first step) using the protein generative model 150 and conditioned on the input 3D representation 504 to generate an updated protein (e.g., updated protein 524). Illustratively, the protein generative model 150 includes a number of update blocks 502 in sequence. Each update block 502 performs similar operations but can include different learned parameter values. The operations of update block 502₁ are shown in detail. Illustratively, the update block 502₁ includes the invariant point attention layer 508, the invariant cross attention layer 510, and the transformer 516. In operation, the update block 502₁ takes as input residue tokens, pair representations, residue frames, ellipsoid tokens, and ellipsoid parameters, and the update block 502₁ generates an updated protein, including updated residue tokens, residue frames, and pair representations. The residue frames each include a translational component and a rotational component, indicating residue points along the protein chain. The pair representations are feature representations indicating the distance between residues, which is associated with the 3D structure of the protein. In some embodiments, the update block 502₁ processes the input residue tokens, pair representations, and residue frames using the invariant point attention layer 508 to generate a result that is added to the residue tokens to generate updated residue tokens; processes the updated residue tokens, the residue frames, and the ellipsoid parameters using the invariant cross attention layer 510 to generate a result that is added to the previously updated residue tokens to generate updated residue tokens; concatenates the updated residue tokens with the ellipsoid tokens; processes the concatenated updated residue tokens and ellipsoid tokens using the transformer 516 that applies a self-attention mechanism to generate updated tokens; splits the updated tokens into updated residue tokens and updated ellipsoid tokens; performs a rigid update using the updated residue tokens and the residue frames to generate updated residue frames; and performs an edge update using the updated residue tokens to generate updated pair representations, according to Algorithm 1 below.

The invariant point attention layer 508 takes as inputs residue tokens specifying the current protein, pair representations, and residue frames, and the invariant point attention layer 508 outputs updated residue tokens. The invariant point attention layer 508 is an attention mechanism between different points along a chain of the protein. The attention is invariant, because the attention remains unchanged regardless of 3D rotational orientation. Any technically feasible invariant point attention layer 508 can be used in some embodiments, such as a pre-trained invariant point attention layer from, e.g., a Multiflow model.

The invariant cross attention layer 510 takes as input the updated residue tokens, the residue frames, and ellipsoid parameters (e.g., the means and covariance matrices) from the 3D representation 504, and the invariant cross attention layer 510 outputs updated residue tokens, shown as updated residue tokens 5141 (referred to herein collectively as updated residue tokens 514 and individually as an updated residue token 514). The invariant cross attention layer 510 performs cross attention between tokens specifying the conditioning information, such as tokens specifying ellipsoid parameters, and the updated residue tokens that specify the protein itself. The cross attention is invariant, because the cross attention remains unchanged regardless of 3D rotational orientation. In some embodiments, the invariant cross attention layer 510 converts the ellipsoid parameters from the 3D representation 504 into rotated coordinate systems of the residues; embeds the converted ellipsoid parameters to generate tokens specifying the ellipsoid parameters; applies a linear layer to the tokens specifying the ellipsoid parameters; adds the result to a sequence representation of the protein; adds the result to a flattened representation of the ellipsoid covariance matrix to which a linear layer is also applied; constructs query, key, and value vectors; and applies attention to the query, key, and value vectors, according to Algorithm 2 below.

The updated residue tokens 514 are concatenated with ellipsoid tokens 5121 (referred to herein collectively as ellipsoid tokens 512 and individually as an ellipsoid token 512) corresponding to ellipsoids in the 3D representation 504 and shown as filled boxes. The concatenation of the updated residue tokens 514 with the ellipsoid tokens 512 is then input into the transformer 516, which applies a self-attention mechanism to generate additional tokens that are split into updated residue tokens 5201 (referred to herein collectively as updated residue tokens 520 and individually as an updated residue token 520) and updated ellipsoid tokens 5181 (referred to herein collectively as updated ellipsoid tokens 518 and individually as an updated ellipsoid token 518). Then, the update block 502₁ updates the residue frames and the residue tokens. In particular, the update block 502₁ performs a rigid update based on the updated residue tokens 522 and the residue frames to generate updated residue frames that include updated translations and orientations of the residues in the protein, and the update block 502₁ performs an edge update based on the updated residue tokens to generate updated pair representations.

More formally, in some embodiments, the iterative integration module 410 can generate proteins that are represented as an array of frames T∈SE(3)^{N}, where each residue's frame Tᵢ=(Rᵢ, tᵢ)∈SE(3) has an associated translation tᵢ ∈ [symbol] ³ and rotation matrix Rᵢ ∈ [symbol] ^{3×3} constructed from backbone coordinates. Additionally, each [symbol] residue has an amino acid type aᵢ ∈{1 . . . 20}. To jointly generate the translations, rotations, and amino acids, the iterative integration module 410 can apply three types of procedures that iteratively update all three modalities. Translations can be handled with linear flow matching from a Gaussian prior, rotations with Riemannian flow matching on SO(3), and residue types with discrete flow matching, resulting in a joint flow that transports from a prior p_0 (t, R, a) to the data distribution p₁(t, R, a) while tracing out a probability path pₜ(t, R, a) where t∈[0,1]. The flow is parameterized by a single backbone architecture with translations, rotations and residue type inputs from which the iterative integration module 410 can predict a time dependent translation vector field

$${v}_{θ , t}^{tr} ( t ) ,$$

rotation vector field

$${v}_{θ , t}^{rot} ( R ) ,$$

and a rate matrix [symbol] _{θ,t} (a) dictating residue type updates. The protein generative model 150 can include several identical update blocks 502, each of which updates d-dimensional residue representations sᵢ∈ [symbol] ^{d} for i∈{1, . . . N}, residue pair representations z_{i,j}∈ [symbol] ^{d} for i,j∈{1 . . . N} and residue frames Tᵢ for i∈{1, . . . N}. The updates are SE(3)-equivariant and can be accomplished with a mixture of shallow transformers and Invariant Point Attention. After all the update blocks 502, the final residue tokens sᵢ and frames Tᵢ can be used to parameterize the flow fields

$${v}_{θ , t}^{tr} ( t ) , {v}_{θ , t}^{rot} ( R ) , {ℛ}_{θ , t} ( a ) .$$

In order to inject ellipsoid conditioning, a pre-trained unconditional model for generating protein strictures, such as a Multiflow model, can be trained to sample p₁(t, R, a) toward sampling an ellipsoid conditioned density p_1 (t, R, a E), thereby obtaining the protein generative model 150. At inference time, ellipsoids can be specified manually or sampled from a second distribution p(E) of novel and diverse ellipsoids to target the density p₁(t, R, a|E)p(E). For fine-tuning, the conditioning information can be provided as additional input. To inject the ellipsoid information, the architecture of the protein generative model 150 includes modifications that minimally perturb the unconditional model at the time of initialization. That is, with an empty set of ellipsoids as input, the untrained conditional model should produce identical outputs as the unconditional model, which can be accomplished by preserving the initial residue representations sᵢ, zᵢⱼ, Tᵢ, and only supplying additional information from 3D ellipsoids to inform their updates. In particular, the protein generative model 150 uses additional tokens eₖ ∈ [symbol] ^{d} for each ellipsoid k∈{1 . . . K} that are of the same dimensionality d as the residue tokens sᵢ. Such tokens are initialized with embeddings of all SE(3)-invariant quantities of ellipsoids-their size nₖ, squared radius of gyration tr Σ, and secondary structure type fₖ. Then, in each layer of the protein generative model 150, these tokens inform the updates of the residue representations sᵢ, zᵢⱼ, Tᵢ (and are themselves updated) via two mechanisms. First, to update the residue tokens sᵢ with information about the locations and shapes of the ellipsoids, the invariant cross attention mechanism of the invariant cross attention layer 510 is used to aggregate values from the ellipsoid tokens in an SE(3)-invariant manner. Similar to invariant point attention, the invariant cross attention layer 510 implements a cross-attention mechanism that uses the residue local frames to enforce invariance, although the ellipsoid tokens are not themselves updated. Second, to provide a mechanism for residue and ellipsoid tokens to mutually update each other, tokens can be concatenated along the sequence dimension right before the transformer 516 stack, and the sequence is re-split after the transformer 516 stack.

In some embodiments, each update block 502 and invariant cross attention layer 510 therein can be implemented according to the pseudo-code of Algorithms 1 and 2, respectively.

### Algorithm 1: Update Block

Input: Residue tokens sᵢ, pair reps zᵢⱼ, residue frames Tᵢ, ellipsoid tokens eₖ,
ellipsoid parameters Eₖ=(μₖ, Σₖ)
s+=InvariantPointAttention(s, z, T)
s+=InvariantCrossAttention(s, T, E)
s←=Concat (s, e)
s+=Transformer(s)
s, e←Split(s)
T←RigidUpdate(s, T)
z+=EdgeUpdate(s)

### Algorithm 2: Invariant Cross Attention

Input: Residue tokens sᵢ and frames Tᵢ=(Rᵢ, tᵢ); ellipsoid parameters Eₖ=(μₖ, Σₖ)

$${r}_{ik} ← {T}_{i}^{- 1} ∘ {μ}_{k} \\ {C}_{ik} ← {R}_{i} {\sum }_{k} {R}_{i}^{T}$$

aᵢₖ=sᵢ+Linear(PosEmbed(rᵢₖ))
a+=Linear (Flatten(Cᵢₖ))
qᵢ=Linear(sᵢ)
kᵢₖ, vᵢₖ=Linear(aᵢₖ)
sᵢ+=Attentionₖ(qᵢ, kiₖ, vᵢₖ)

In addition to fine-tuning the protein generative model 150 that samples p_{θ}(t, R, a)≈p₁(t, R, a) to obtain the distribution p_{θ}(t, R, a|E), the two distributions can be interpolated via classifier-free guidance controlled by a guidance parameter λ≥0. Doing so enables finding the optimal λ to trade off between the designability of p_{θ}(t, R, a) that is recovered with λ=0 and the diversity, novelty, and ellipsoid adherence of p_{θ}(t,R, a|E) corresponding to λ=1. In some embodiments, the joint flow over translations, rotations, and discrete residue types can be guided by separately interpolating their flow fields at each inference step as follows. Translations can be interpolated by interpolating the unconditional vector field

$${v}_{θ , t}^{tr} ( t )$$

and the conditioned version

$${v}_{θ , t ,}^{tr} ( t , E ) as {λv}_{θ , t}^{tr} ( t , E ) + ( 1 - λ ) {v}_{θ , t}^{tr} ( t ) .$$

Since the conditional probability paths for translations are Gaussian paths, such an interpolation corresponds to guided flows that sample the same approximation of the unconditional distribution tilted by the conditional distribution as guided diffusion models, i.e., an approximation of p₁(t)^{(1-λ)p}₁(t|E)^{λ}, if models that only sample translations were interpolated. Rotations can be interpolated in analogy to translations, as

$$λ {v}_{θ , t}^{rot} ( t , E ) + ( 1 - λ ) {v}_{θ , t}^{rot} ( t ) .$$

Discrete flow can be interpolated by constructing the rate matrix for the discrete flow as the expectation of the conditional rate matrix over predicted probabilities of the denoised residues obtained as a combination of the unconditional model predictions and the ellipsoid conditioned model predictions. Specifically, the unconditionally predicted probabilities can be tilted by the ellipsoid conditioned probabilities

$${{p}_{θ} ( {a}_{i}^{( 1 )} \vert {a}_{i}^{( t )} )}^{( 1 - λ )} {{p}_{θ} ( {a}_{i}^{( 1 )} \vert {a}_{i}^{( t )} , E )}^{λ} ,$$

where the superscript denotes denoising time.

During inference, the unconditional model p_{θ}(t, R, a) produces the selfconditioning variable X, and from the ellipsoid conditioned model p_{θ}(t, R, a|E), X_{E} can be obtained. Instead of supplying X to the unconditional and X_{E} to the conditioned model, λX_{E}+(1−λ)X can be used for both, which achieves better designability and ellipsoid adherence for all λ.

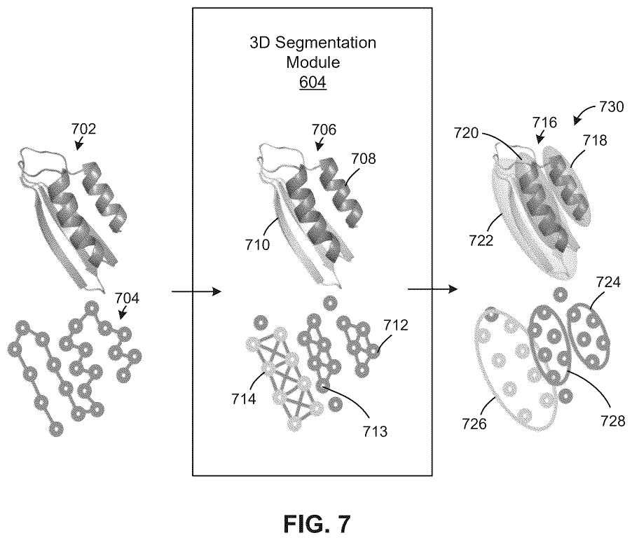

FIG. 6 is a more detailed illustration of the model trainer 116 of FIG. 1, according to various embodiments. As shown, the model trainer 116 includes, without limitation, an ellipsoid segmentation module 604 and a supervised learning module 608. In operation, the model trainer 116 receives as input proteins 602. The proteins 602 can include known proteins from, for example, a library of proteins. The ellipsoid segmentation module 604 segments the proteins to generate 3D representations 606, such as ellipsoid representations. The ellipsoid segmentation module 604 is discussed in greater detail below in conjunction with FIG. 7. The supervised learning module 608 performs supervised learning using the 3D representations 606 and the proteins 602 as training data to generate the protein generative model 150.

As described, flow matching aims to learn a time-dependent vector field v_{θ,t} that, when integrated from a start time t=0 to t=1, transports samples from a noise distribution x₀˜p₀ to a data distribution x₁˜p₁. To train v_{θ,t}, the model trainer 116 can sample partially noised data from a conditional probability path pₜ(x|x₀, x₁) satisfying p₀(x|x₀, x₁)≈δ(x−x₀) and p₁(x|x₀, x₁)≈δ(x−x₁), such as a Dirac that traces out a straight line between x₀ and x₁ or a geodesic for flow matching on manifolds. At the sampled noisy datapoints xₜ, the model trainer 116 evaluates the vector field v_{θ,t}(Xₜ) and regresses the vector field against the conditional vector field uₜ(Xₜ|x₀, x₁) that corresponds to the conditional probability path through the continuity equation

$$\frac{\partial }{\partial t} {p}_{t} = - ∇ \cdot ( {p}_{t} {u}_{t} ) .$$

At convergence, v_{θ,t} approximates the marginal vector field uₜ(x) (since the gradients are equivalent to regressing against uₜ(x)) that evolves the prior p₀ to the data distribution p₁ through the marginal probability path pₜ(x)=∫pₜ(x|x₀, x₁)p₀(x₀)p₁(x₁)dx₀dx₁.

FIG. 7 illustrates how an exemplar ellipsoid representation can be generated from a known protein, according to various embodiments. As shown, the ellipsoid segmentation module 604 of the model trainer 116 can segment a protein, shown as protein 702, using semantic graph clustering and fit Gaussians to the clusters to generate a 3D representation, shown as ellipsoid representation 730 that includes ellipsoids 718, 720, and 722. The 3D representations (e.g., ellipsoid representation 730) can then be used to train the protein generative model 150.

Illustratively, the ellipsoid segmentation module 604 takes as input a protein 702, such as a known protein from a library of proteins. Given the input protein 702, the ellipsoid segmentation module 604 generates a 3D ellipsoid representation in two steps: segmentation of the protein into semantically coherent regions, and extraction of ellipsoid descriptions (μₖ, Σₖ, nₖ, fₖ) for each region.

In some embodiments, the ellipsoid segmentation module 604 can perform the first step of segmenting the protein using semantic graph clustering by placing two residues in the same region if and only if the residues are both spatially proximal and semantically similar. In such cases, the ellipsoid segmentation module 604 can construct a segmentation graph by drawing an edge for each such pair of residues and determine the list of connected components of the segmentation graph. Illustratively, the ellipsoid segmentation module 604 annotates and draws edges between residues that are determined to (1) have the same feature, and (2) be within a predefined distance of each other, such as 5 Å. In some embodiments, having the same feature can include being part of a same secondary structure, such as an alpha helix or a beta sheet. Illustratively, annotations 712, 713, and 714 indicate residues of the protein 702 that belong to the same secondary structures. In some other embodiments, having the same feature includes having the same functionality (e.g., functionality of a functionally relevant site) or any other technically feasible property (e.g., electron density). In the case of 3D ellipsoids specifying a secondary structure layout, i.e., regions of alpha helices and beta sheets, the feature space is a two-class space of secondary structure types fₖ ∈ [symbol] ={α,β}. In such cases, the ellipsoid segmentation module 604 can featurize residues using, e.g., the DSSP (Dictionary of Secondary Structure of Proteins) algorithm, and draw edges in the segmentation graph between amino acids with the same secondary structure label and within a certain distance, such as 5 Å.

In some embodiments the ellipsoid segmentation module 604 can perform the second step of extracting ellipsoid descriptions for each region by aggregating the residue features in the region to obtain fₖ and computing the mean and covariance of the Cα positions in order to fit a Gaussian (e.g., Gaussians 726, 724, and 728) to the connected residues in the region. The Gaussians can also be converted to ellipsoids using, for example, a predefined Mahalanobis distance (e.g., a Mahalanobis distance of \sqrt{5}) to define the boundaries of the ellipsoids. Illustratively, the Gaussians 726, 724, and 728 can be converted to the ellipsoids 722, 718, and 720 in the ellipsoid representation 730, respectively. In addition, the ellipsoids can inherit the annotation fₖ from the label of constituent residues inside the ellipsoids. In some embodiments, loop residues and ellipsoids with fewer than a predefined number of residues (e.g., 5 residues) can be excluded from the ellipsoid representation.

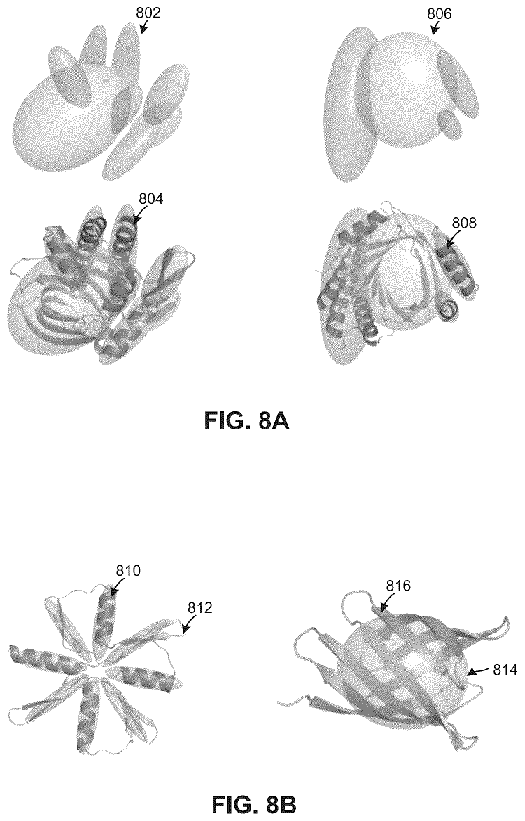

FIG. 8A illustrates exemplar proteins generated based on ellipsoid representations generated by the statistical model 408, according to various embodiments. As shown, the 3D representation generator 406 of the protein generating application 146 can use the statistical model 408 to generate 3D ellipsoid representations 802 and 806 that include ellipsoids specifying the locations of beta sheets and alpha helices, as described above in conjunction with FIG. 4. Then, the iterative integration module 410 of the protein generating application 146 can generate proteins 804 and 808 that include beta sheets and alpha helices in the locations specified by the 3D ellipsoid representations 802 and 806, respectively.

FIG. 8B illustrates exemplar proteins generated based on user-specified ellipsoid representations, according to various embodiments. As shown, given a user-specified ellipsoid representation 810 that uses ellipsoids to specify the locations of beta sheets and alpha helices, the protein generating application 146 can generate a protein 812 that includes beta sheets and alpha helices in the specified locations. Similarly, given a user-specified ellipsoid representation 814 that uses an ellipsoid to specify the location of a beta barrel, the protein generating application 146 can generate a protein 816 that includes a beta barrel that conforms to the ellipsoid representation 814.

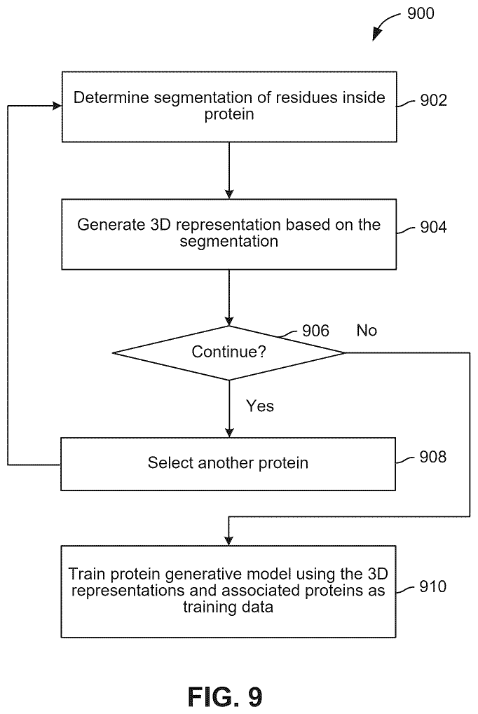

FIG. 9 is a flow diagram of method steps for training a protein generative model, according to various embodiments. Although the method steps are described in conjunction with the systems of FIGS. 1-7, persons skilled in the art will understand that any system configured to perform the method steps in any order falls within the scope of the present embodiments.

As shown, a method 900 begins at step 902, where the model trainer 116 determines a segmentation of residues inside a protein. As described, in some embodiments, the model trainer 116 can segment the protein using semantic graph clustering by placing two residues in the same region if and only if the residues are both spatially proximal and semantically similar. In such cases, the model trainer 116 can construct a segmentation graph by drawing an edge for each such pair of residues and determine the list of connected components of the segmentation graph. As described above in conjunction with FIG. 7, in some embodiments, the model trainer 116 annotates and draws edges between residues that are determined to (1) have the same feature, and (2) be within a predefined distance of each other, such as 5 Å. In some embodiments, having the same feature can include being part of a same secondary structure, such as an alpha helix or a beta sheet. In some other embodiments, having the same feature includes having the same functionality (e.g., functionality of a functionally relevant site) or any other technically feasible property (e.g., electron density). In the case of 3D ellipsoids specifying a secondary structure layout, i.e., regions of alpha helices and beta sheets, the feature space is a two-class space of secondary structure types fₖ ∈ [symbol] ={α,β}. In such cases, the model trainer 116 can featurize residues using, e.g., the DSSP (Dictionary of Secondary Structure of Proteins) algorithm, and draw edges in the segmentation graph between amino acids with the same secondary structure label and within a certain distance, such as 5 Å.

At step 904, the model trainer 116 generates a 3D representation based on the segmentation. As described above in conjunction with FIG. 7, in some embodiments, when the 3D representation is a 3D ellipsoid representation, the model trainer 116 can fit Gaussians to the clusters determined using semantic graph clustering. In such cases, the model trainer 116 can extract ellipsoid descriptions for each region by aggregating the residue features in regions and fitting a Gaussian to the connected residues in the region. In such cases, the Gaussians can also be converted to ellipsoids using, for example, a predefined Mahalanobis distance (e.g., a Mahalanobis distance of \sqrt{5}) to define the boundaries of the ellipsoids. In addition, the ellipsoids can inherit the annotation fₖ from the label of constituent residues inside the ellipsoids. In some embodiments, loop residues and ellipsoids with fewer than a predefined number of residues (e.g., 5 residues) can be excluded from the ellipsoid representation.

At step 906, if the model trainer 116 determines to continue generating 3D representations, then the method 900 continues to step 908, where the model trainer 116 selects another protein. The model trainer 116 can iteratively process any number of proteins, such as the proteins within a library of proteins, in some embodiments.

On the other hand, if the model trainer 116 determines to stop generating 3D representations, then the method 900 proceeds directly to step 910, where the model trainer 116 trains a protein generative model (e.g., protein generative model 150) using the 3D representations and associated proteins as training data. In some embodiments, the protein generative model can be trained via a flow matching technique that learns a time-dependent vector field v_{θ,t} that, when integrated from a start time t=0 to t=1, transports samples from a noise distribution x₀˜p₀ to a data distribution x₁˜p₁. In such cases, the model trainer 116 can train the protein generative model as described above in conjunction with FIG. 6.

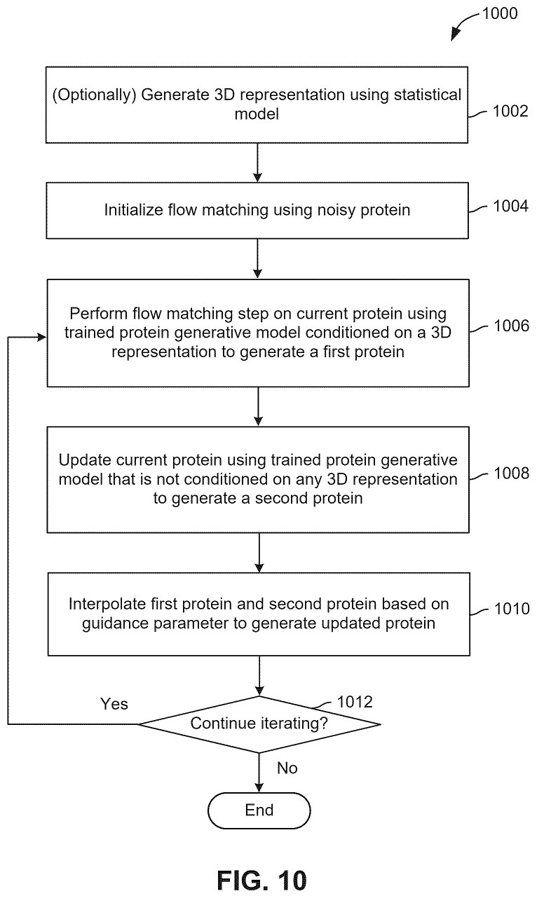

FIG. 10 is a flow diagram of method steps for generating proteins using a trained protein generative model, according to various embodiments. Although the method steps are described in conjunction with the systems of FIGS. 1-7, persons skilled in the art will understand that any system configured to perform the method steps in any order falls within the scope of the present embodiments.

As shown, a method 1000 begins at step 1002, where the protein generating application 146 optionally generates a 3D representation using the statistical model 408. In some embodiments, the 3D representation can include a set of ellipsoids and associated annotations that specify secondary structures, functionalities, and/or other properties associated with the ellipsoids. In some embodiments, when no user-specified 3D representation is received as input, the protein generating application 146 can automatically generate a 3D representation using the statistical model 408. For example, in some embodiments, the protein generating application 146 can automatically generate 3D ellipsoid representations by randomly sampling means for a set of ellipsoids, with randomly assigned annotations, from a distribution (e.g., a Gaussian distribution) and randomly sampling covariance matrices for the set of ellipsoids from another distribution (e.g., a Wishart distribution that is a distribution over plausible covariance matrices), while penalizing configurations in which ellipsoids overlap significantly, as described above in conjunction with FIG. 4. As described, in some embodiments, the annotations for the ellipsoids can indicate secondary structures, such as alpha sheets and/or beta helices. In some embodiments, the annotations for the ellipsoids can indicate functionalities, such as a binding site. In some embodiments, the annotations for the ellipsoids can indicate other properties, such as electron density. For example, experimentally measured electron density from cryo-electron microcopy (CryoEM) could be used to guide the generation of proteins, allowing atomistic model building from CryoEM density maps.

At step 1004, the protein generating application 146 initializes the iterative updating using a noisy protein. In some embodiments, the noisy protein can be generated to include residues having random positions and orientations. Each residue is an amino acid that can be included in a protein.

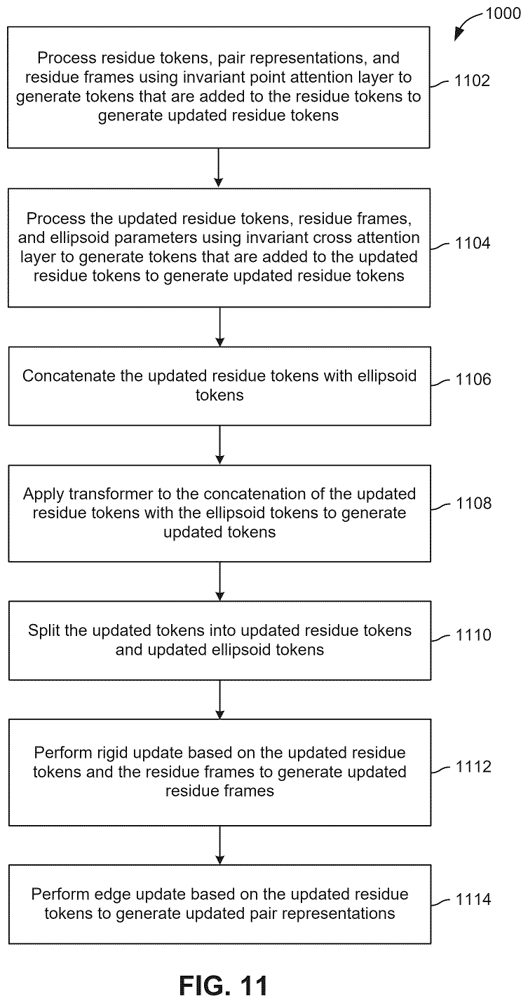

At step 1006, the protein generating application 146 performs an update step on a current protein to integrate the neural network-defined vector field using the trained protein generative model 150, which is the neural network, conditioned on a 3D representation to generate an updated protein. The 3D representation can be either automatically generated at step 1002 or input by a user via, . . . , a user interface. In some embodiments, the protein generative model 150 can be trained according to the method 900 described above in conjunction with FIG. 9. In some embodiments, the update step includes processing a current protein using the protein generative model 150 and conditioned on the input 3D representation 504 to generate an updated protein. The protein generative model 150 includes a number of update blocks (e.g., update blocks 502) in sequence. In operation, an update block takes as input residue tokens, pair representations, residue frames, ellipsoid tokens, and ellipsoid parameters, and the update block generates an updated protein, including updated residue tokens, residue frames, and pair representations. As discussed in greater detail below in conjunction with FIG. 11, in some embodiments, the update block processes the input residue tokens, pair representations, and residue frames using an invariant point attention layer (e.g., invariant point attention layer 508) to generate a result that is added to the residue tokens to generate updated residue tokens; processes the updated residue tokens, the residue frames, and the ellipsoid parameters using an invariant cross attention layer (e.g., invariant cross attention layer 510) according to Algorithm 2 to generate a result that is added to the previously updated residue tokens to generate updated residue tokens; concatenates the updated residue tokens with the ellipsoid tokens; processes the concatenated updated residue tokens and ellipsoid tokens using a transformer (e.g., transformer 516) that applies a self-attention mechanism to generate updated tokens; splits the updated tokens into updated residue tokens and updated ellipsoid tokens; performs a rigid update using the updated residue tokens and the residue frames to generate updated residue frames; and performs an edge update using the updated residue tokens to generate updated pair representations, according to Algorithm 1.

In some embodiments, the protein generating application 146 also updates the current protein using the trained protein generative model 150 that is not conditioned on any 3D representation. In such cases, the protein generating application 146 can interpolate the conditional and unconditional vector fields based on a guidance parameter to generate an updated protein, as described above in conjunction with FIG. 5. For example, in some embodiments, the guidance parameter can be set so that the first and second vector fields are combined in a manner that overemphasizes the conditioning. More specifically, in some embodiments, the guidance parameter can be tuned (e.g., by a user) to obey the 3D representation conditioning to a greater or a lesser degree.

At step 1008, if the protein generating application 146 determines to continue iterating, then the method 1000 returns to step 1006, where the protein generating application 146 performs another update step on the current protein using the trained protein generative model 150 conditioned on the 3D representation to generate another protein. In some embodiments, the protein generating application 146 can iterate for a predefined number of iterations.

If, on the other hand, the protein generating application 146 determines to stop iterating, then the method 1000 ends. In some embodiments, the protein generation application 146 can perform a predefined number of update steps, such as 100 steps.

FIG. 11 is a flow diagram of method steps for performing an update block within the trained protein generative model 150, according to various embodiments. Although the method steps are described in conjunction with the systems of FIGS. 1-7, persons skilled in the art will understand that any system configured to perform the method steps in any order falls within the scope of the present embodiments.

As shown, a method 1100 begins at step 1102, where the protein generative model 150 processes residue tokens, pair representations, and residue frames using an invariant point attention layer (e.g., invariant point attention layer 508) to generate tokens that are added to the residue tokens to generate updated residue tokens. The invariant point attention layer is an attention mechanism between different points along a chain of the protein. The attention is invariant, because the attention remains unchanged regardless of 3D rotational orientation. Any technically feasible invariant point attention layer can be used in some embodiments, such as a pre-trained invariant point attention layer from, e.g., a Multiflow model.

At step 1004, the protein generative model 150 processes the updated residue tokens, residue frames, and ellipsoid parameters using an invariant cross attention layer (e.g., invariant cross attention layer 510) to generate tokens that are added to the updated residue tokens from step 1102 to generate updated residue tokens. As described above in conjunction with FIG. 5, in some embodiments, the invariant cross attention layer performs cross attention between tokens specifying the conditioning information, such as tokens specifying ellipsoid parameters, and the updated residue tokens that specify the protein itself. The cross attention is invariant, because the cross attention remains unchanged regardless of 3D rotational orientation. In some embodiments, the invariant cross attention layer converts the ellipsoid parameters from a 3D representation into rotated coordinate systems of the residues; embeds the converted ellipsoid parameters to generate tokens specifying the ellipsoid parameters; applies a linear layer to the tokens specifying the ellipsoid parameters; adds the result to a sequence representation of the protein; adds the result to a flattened representation of the ellipsoid covariance matrix to which a linear layer is also applied; constructs query, key, and value vectors; and applies attention to the query, key, and value vectors, according to Algorithm 2.

At step 1006, the protein generative model 150 concatenates the updated residue tokens generated at step 1004 with ellipsoid tokens. In some embodiments, to provide a mechanism for residue and ellipsoid tokens to mutually update each other, tokens can be concatenated along the sequence dimension before a transformer stack, and the sequence is re-split after the transformer stack, as described above in conjunction with FIG. 5.

At step 1008, the protein generative model 150 applies a transformer to the concatenation of the updated residue tokens with the ellipsoid tokens to generate updated tokens. The transformer applies a self-attention mechanism to generate updated tokens.

At step 1010, the protein generative model 150 splits the updated tokens generated at step 1008 into updated residue tokens and updated ellipsoid tokens. Then, at step 1012, the protein generative model 150 performs a rigid update based on the updated residue tokens and the residue frames to generate updated residue frames. In addition, at step 1014, the protein generative model 150 performs an edge update based on the updated residue tokens to generate updated pair representations.

Embodiments of the present disclosure provide techniques for generating proteins conditioned on 3D representations. In some embodiments, a 3D representation includes one or more shapes, such as one or more ellipsoids, specifying the locations of one or more annotated portions of a protein, such as the locations of secondary structures (e.g., alpha helices and/or beta sheets) within the protein or the locations of portions of the protein having certain functionalities or other properties. A user can specify a 3D representation to use for generating a protein. Alternatively, a protein generating application can automatically generate a 3D representation using a statistical model. In the case of a 3D representation that includes ellipsoids, the statistical model can randomly sample means and covariance matrices for a number of ellipsoids, while penalizing configurations in which ellipsoids overlap. The protein generating application generates a protein conditioned on a user-specified or automatically generated 3D representation by iteratively integrating a vector field defined by a neural network that is learned via a flow matching technique. The flow matching technique learns a flow that can be described through a differential equation or continuous time Markov chain, which the protein generating application can then numerically solve in a step-wise manner by sampling the neural network. The generated protein includes a sequence of residues and a structure conforming to the 3D representation. The iterative integration of the vector field includes, for multiple time steps, processing a current protein and the 3D representation using a trained protein generative model to generate an updated protein. By performing multiple such iterative steps, a protein that begins as random noise can be transformed into a protein that conforms to the 3D representation. The protein generative model is the neural network that includes, among other things, an invariant cross attention that allows tokens corresponding to the protein to attend to tokens corresponding to the 3D representation. In some embodiments, generating a protein can include interpolating a conditional vector field that is conditioned on a 3D representation and an unconditional vector field based on a guidance parameter.

To train the protein generative model, a model trainer performs 3D segmentation on a number of proteins, such as proteins from a library of proteins, and the model trainer generates 3D representations based on the 3D segmentations. For example, in some embodiments, the model trainer can segment the proteins into secondary structures or based on the functionality or other properties of portions of the proteins, fit Gaussians to the segmented portions of the proteins, and convert the Gaussians to ellipsoids using, for example, a predefined Mahalanobis distance to define boundaries of the ellipsoids. Then, the model trainer can train the protein generative model using a flow matching technique and the 3D representations and corresponding proteins as training data.

At least one technical advantage of the disclosed techniques relative to the prior art is the disclosed techniques permit users to control the 3D spatial layouts of proteins that are generated by a trained machine learning model. In particular, the 3D spatial layouts can be controlled using 3D ellipsoid representations that are informative enough to control the generation of diverse proteins, while being human-interpretable and easy to construct, such as through sketches of ellipsoids in the ellipsoid representations. As the function of a protein depends on the structure of the protein, being able to explicitly control the 3D spatial layouts of generated proteins according to techniques disclosed herein permits the generated proteins to exhibit desired properties to a higher degree than proteins that are generated according to prior art approaches. These technical advantages represent one or more technological improvements over prior art approaches.
1. In some embodiments, a computer-implemented method for generating proteins comprises generating, using a trained machine learning model, a first protein based on a three-dimensional (3D) representation of a spatial layout for the first protein, wherein generating the first protein comprises applying cross-attention between one or more first tokens associated with the 3D representation and one or more second tokens associated with a second protein.
2. The computer-implemented method of clause 1, wherein generating the first protein comprises performing one or more update steps to integrate a vector field defined by the trained machine learning model.
3. The computer-implemented method of clauses 1 or 2, wherein the cross-attention is invariant to 3D rotational orientation.
4. The computer-implemented method of any of clauses 1-3, wherein applying the cross-attention comprises converting one or more parameters associated with the 3D representation into one or more rotated coordinate systems of one or more residues of the second protein to generate one or more converted parameters, performing a position embedding of the one or more converted parameters to generate the one or more first tokens, generating one or more third tokens by adding a sequence representation of the second protein to the one or more first tokens after applying a first linear layer to the one or more first tokens, adding the one or more third tokens to a flattened representation of the one or more converted parameters after applying a second linear layer to the one or more converted parameters to generate one or more fourth tokens, determining a query vector based on the sequence representation after applying a third linear layer to the sequence representation, determining a key vector and a value vector based on the one or more fourth tokens, and applying attention between the query, key, and value vectors to generate one or more fifth tokens.
5. The computer-implemented method of any of clauses 1-4, wherein the trained machine learning model comprises one or more first layers that apply attention between one or more points associated with the second protein, one or more second layers that apply the cross-attention between the one or more first tokens and the one or more second tokens to generate one or more third tokens, and a transformer that generates one or more fourth tokens based on the one or more third tokens and one or more fifth tokens associated with the 3D representation.
6. The computer-implemented method of any of clauses 1-5, wherein the 3D representation comprises one or more ellipsoids and one or more annotations associated with the one or more ellipsoids.
7. The computer-implemented method of any of clauses 1-6, wherein each annotation included in the one or more annotations specifies at least one of a secondary structure, a functionality, or a property of a corresponding ellipsoid included in the one or more ellipsoids.
8. The computer-implemented method of any of clauses 1-7, further comprising either receiving the 3D representation via a user interface or generating the 3D representation based on a statistical model that samples at least one parameter associated with the 3D representation.
9. The computer-implemented method of any of clauses 1-8, wherein generating the first protein comprises combining, based on a guidance parameter, a first vector field conditioned on the 3D representation and a second vector field not conditioned on the 3D representation.
10. The computer-implemented method of any of clauses 1-9, further comprising segmenting a plurality of proteins based on at least one of secondary structures, functionalities, or properties associated with portions of the plurality of proteins to generate a plurality of segmentations, fitting ellipsoids to the plurality of segmentations to generate a plurality of ellipsoid representations, and performing one or more flow matching operations to train an untrained machine learning model based on the plurality of ellipsoid representations to generate the trained machine learning model.
11. In some embodiments, one or more non-transitory computer-readable media store instructions that, when executed by at least one processor, cause the at least one processor to perform the steps of generating, using a trained machine learning model, a first protein based on a three-dimensional (3D) representation of a spatial layout for the first protein, wherein generating the first protein comprises applying cross-attention between one or more first tokens associated with the 3D representation and one or more second tokens associated with a second protein.
12. The one or more non-transitory computer-readable media of clause 11, wherein generating the first protein comprises performing one or more update steps to integrate a vector field defined by the trained machine learning model.
13. The one or more non-transitory computer-readable media of clauses 11 or 12, wherein applying the cross-attention comprises converting one or more parameters associated with the 3D representation into one or more rotated coordinate systems of one or more residues of the second protein to generate one or more converted parameters, performing a position embedding of the one or more converted parameters to generate the one or more first tokens, generating one or more third tokens by adding a sequence representation of the second protein to the one or more first tokens after applying a first linear layer to the one or more first tokens, adding the one or more third tokens to a flattened representation of the one or more converted parameters after applying a second linear layer to the one or more converted parameters to generate one or more fourth tokens, determining a query vector based on the sequence representation after applying a third linear layer to the sequence representation, determining a key vector and a value vector based on the one or more fourth tokens, and applying attention between the query, key, and value vectors to generate one or more fifth tokens.
14. The one or more non-transitory computer-readable media of any of clauses 11-13, wherein the trained machine learning model comprises one or more first layers that apply attention between one or more points associated with the second protein, one or more second layers that apply the cross-attention between the one or more first tokens and the one or more second tokens to generate one or more third tokens, and a transformer that generates one or more fourth tokens based on the one or more third tokens and one or more fifth tokens associated with the 3D representation.
15. The one or more non-transitory computer-readable media of any of clauses 11-14, wherein the trained machine learning model further comprises a first module that performs a rigid update to one or more residue frames associated with the second protein based on one or more sixth tokens included in the one or more fifth tokens, and a second module that performs an edge update to one or more pair representations associated with the second protein based on the one or more sixth tokens.
16. The one or more non-transitory computer-readable media of any of clauses 11-15, wherein the 3D representation comprises one or more ellipsoids and one or more annotations associated with the one or more ellipsoids.
17. The one or more non-transitory computer-readable media of any of clauses 11-16, wherein each annotation included in the one or more annotations specifies at least one of a secondary structure, a functionality, or a property of a corresponding ellipsoid included in the one or more ellipsoids.
18. The one or more non-transitory computer-readable media of any of clauses 11-17, wherein the 3D representation comprises one or more ellipsoids, and wherein the instructions, when executed by the at least one processor, further cause the at least one processor to perform the step of generating the 3D representation based on a statistical model that samples at least one parameter associated with the one or more ellipsoids and penalizes overlapping ellipsoids.
19. The one or more non-transitory computer-readable media of any of clauses 11-18, wherein the first protein comprises a sequence of residues and a structure.
20. In some embodiments, a system comprises one or more memories storing instructions, and one or more processors that are coupled to the one or more memories and, when executing the instructions, are configured to generate, using a trained machine learning model, a first protein based on a three-dimensional (3D) representation of a spatial layout for the first protein, wherein generating the first protein comprises applying cross-attention between one or more first tokens associated with the 3D representation and one or more second tokens associated with a second protein.

Any and all combinations of any of the claim elements recited in any of the claims and/or any elements described in this application, in any fashion, fall within the contemplated scope of the present disclosure and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A computer-implemented method for generating proteins, the method comprising:
generating, using a trained machine learning model, a first protein based on a three-dimensional (3D) representation of a spatial layout for the first protein,
wherein generating the first protein comprises applying cross-attention between one or more first tokens associated with the 3D representation and one or more second tokens associated with a second protein.

2. The computer-implemented method of claim 1, wherein generating the first protein comprises performing one or more update steps to integrate a vector field defined by the trained machine learning model.

3. The computer-implemented method of claim 1, wherein the cross-attention is invariant to 3D rotational orientation.

4. The computer-implemented method of claim 1, wherein applying the cross-attention comprises:
converting one or more parameters associated with the 3D representation into one or more rotated coordinate systems of one or more residues of the second protein to generate one or more converted parameters;
performing a position embedding of the one or more converted parameters to generate the one or more first tokens;
generating one or more third tokens by adding a sequence representation of the second protein to the one or more first tokens after applying a first linear layer to the one or more first tokens;
adding the one or more third tokens to a flattened representation of the one or more converted parameters after applying a second linear layer to the one or more converted parameters to generate one or more fourth tokens;
determining a query vector based on the sequence representation after applying a third linear layer to the sequence representation;
determining a key vector and a value vector based on the one or more fourth tokens; and
applying attention between the query, key, and value vectors to generate one or more fifth tokens.

5. The computer-implemented method of claim 1, wherein the trained machine learning model comprises:
one or more first layers that apply attention between one or more points associated with the second protein;
one or more second layers that apply the cross-attention between the one or more first tokens and the one or more second tokens to generate one or more third tokens; and
a transformer that generates one or more fourth tokens based on the one or more third tokens and one or more fifth tokens associated with the 3D representation.

6. The computer-implemented method of claim 1, wherein the 3D representation comprises one or more ellipsoids and one or more annotations associated with the one or more ellipsoids.

7. The computer-implemented method of claim 6, wherein each annotation included in the one or more annotations specifies at least one of a secondary structure, a functionality, or a property of a corresponding ellipsoid included in the one or more ellipsoids.

8. The computer-implemented method of claim 1, further comprising either receiving the 3D representation via a user interface or generating the 3D representation based on a statistical model that samples at least one parameter associated with the 3D representation.

9. The computer-implemented method of claim 1, wherein generating the first protein comprises combining, based on a guidance parameter, a first vector field conditioned on the 3D representation and a second vector field not conditioned on the 3D representation.

10. The computer-implemented method of claim 1, further comprising:
segmenting a plurality of proteins based on at least one of secondary structures, functionalities, or properties associated with portions of the plurality of proteins to generate a plurality of segmentations;
fitting ellipsoids to the plurality of segmentations to generate a plurality of ellipsoid representations; and
performing one or more flow matching operations to train an untrained machine learning model based on the plurality of ellipsoid representations to generate the trained machine learning model.

11. One or more non-transitory computer-readable media storing instructions that, when executed by at least one processor, cause the at least one processor to perform the steps of:
generating, using a trained machine learning model, a first protein based on a three-dimensional (3D) representation of a spatial layout for the first protein,
wherein generating the first protein comprises applying cross-attention between one or more first tokens associated with the 3D representation and one or more second tokens associated with a second protein.

12. The one or more non-transitory computer-readable media of claim 11, wherein generating the first protein comprises performing one or more update steps to integrate a vector field defined by the trained machine learning model.

13. The one or more non-transitory computer-readable media of claim 11, wherein applying the cross-attention comprises:
converting one or more parameters associated with the 3D representation into one or more rotated coordinate systems of one or more residues of the second protein to generate one or more converted parameters;
performing a position embedding of the one or more converted parameters to generate the one or more first tokens;
generating one or more third tokens by adding a sequence representation of the second protein to the one or more first tokens after applying a first linear layer to the one or more first tokens;
adding the one or more third tokens to a flattened representation of the one or more converted parameters after applying a second linear layer to the one or more converted parameters to generate one or more fourth tokens;
determining a query vector based on the sequence representation after applying a third linear layer to the sequence representation;
determining a key vector and a value vector based on the one or more fourth tokens; and
applying attention between the query, key, and value vectors to generate one or more fifth tokens.

14. The one or more non-transitory computer-readable media of claim 11, wherein the trained machine learning model comprises:
one or more first layers that apply attention between one or more points associated with the second protein;
one or more second layers that apply the cross-attention between the one or more first tokens and the one or more second tokens to generate one or more third tokens; and
a transformer that generates one or more fourth tokens based on the one or more third tokens and one or more fifth tokens associated with the 3D representation.

15. The one or more non-transitory computer-readable media of claim 14, wherein the trained machine learning model further comprises:
a first module that performs a rigid update to one or more residue frames associated with the second protein based on one or more sixth tokens included in the one or more fifth tokens; and
a second module that performs an edge update to one or more pair representations associated with the second protein based on the one or more sixth tokens.

16. The one or more non-transitory computer-readable media of claim 11, wherein the 3D representation comprises one or more ellipsoids and one or more annotations associated with the one or more ellipsoids.

17. The one or more non-transitory computer-readable media of claim 16, wherein each annotation included in the one or more annotations specifies at least one of a secondary structure, a functionality, or a property of a corresponding ellipsoid included in the one or more ellipsoids.

18. The one or more non-transitory computer-readable media of claim 11, wherein the 3D representation comprises one or more ellipsoids, and wherein the instructions, when executed by the at least one processor, further cause the at least one processor to perform the step of generating the 3D representation based on a statistical model that samples at least one parameter associated with the one or more ellipsoids and penalizes overlapping ellipsoids.

19. The one or more non-transitory computer-readable media of claim 11, wherein the first protein comprises a sequence of residues and a structure.

20. A system, comprising:
one or more memories storing instructions; and
one or more processors that are coupled to the one or more memories and, when executing the instructions, are configured to:
generate, using a trained machine learning model, a first protein based on a three-dimensional (3D) representation of a spatial layout for the first protein,
wherein generating the first protein comprises applying cross-attention between one or more first tokens associated with the 3D representation and one or more second tokens associated with a second protein.
